(19) United States
(12) Patent Application Publication
(10) Pub. No.: US 2010/0280004 A1
(43) Pub. Date: Nov. 4, 2010

(54) SMALL MOLECULE CORRECTORS OF DELTAF508 CFTR TRAFFICKING

(75) Inventors: Raymond Andersen, Vancouver (CA); Robert Keyzers, Glen Osmond (AU); Christopher Anthony Gray, Saint John (CA); David Y. Thomas, Montreal West (CA); John W. Hanrahan, Montreal West (CA); Graeme W. Carlile, Ville St-Laurent (CA)

Correspondence Address: FENWICK & WEST LLP, SILICON VALLEY CENTER, 801 CALIFORNIA STREET, MOUNTAIN VIEW, CA, 94041, US

(73) Assignees: The University of British Columbia and The Royal Institution for the Advancement of; Learning/McGill University

(21) Appl. No.: 12/667,284

(22) PCT Filed: Jul. 3, 2008
(86) PCT No.: PCT/CA08/01238

§ 371 Date: Jun. 18, 2010

Related U.S. Application Data
(60) Provisional application No. 60/929,562, filed on Jul. 3, 2007.

(51) Int. Cl. A61K 31/55; C07D 487/04; A61P 5/00

(52) U.S. Cl. 514/212.06; 540/521

(57) ABSTRACT

This invention provides compounds of Formula I. The compounds are correctors of ΔF508 CFTR trafficking. Also provided are uses of compounds of Formula I for treatment, as well as methods of treatment, of cystic fibrosis.

### TECHNICAL FIELD

This invention relates to small molecules correctors of DELTAF508 (ΔF508) CFTR trafficking and more specifically to latonduine related molecules and analogues. The small molecules may be used in the treatment of cystic fibrosis or in the methods of treatment for cystic fibrosis.

### BACKGROUND OF THE INVENTION

Cystic Fibrosis (CF) is the most common lethal genetic disease affecting Caucasians. It is an autosomal recessive disease and occurs with a frequency of one in 2200 live births in North America and Europe. CF affects epithelial cells that line the airways, intestine and exocrine tissues such as the sweat glands and pancreatic ducts. The airway mucus of CF patients becomes viscous and dehydrated, disrupting the mucociliary clearance of inhaled pathogens. This leads to recurring infections in the airways by pathogens such as, Pseudomonas aeruginosa, Hemophilus influenzae and Staphylococcus aureus. The resulting inflammation causes fibrosis and a gradual deterioration in lung function resulting in a shortening of mean life span for CF patients to 37 years.

CF is caused by mutations in the cystic fibrosis transmembrane conductance regulator (CFTR) gene. This gene encodes a 1480 amino acid protein that functions primarily as a chloride ion channel in the plasma membrane of epithelial cells. Over 1430 different mutations have been documented in the CFTR gene, but by far the most common mutation is a phenylalanine deletion (ΔF508) in the first nucleotide binding domain (NBD), which is present in at least one allele in ˜90% of CF patients.

The ΔF508 CFTR protein is firstly retained in the endoplasmic reticulum (ER) prior to degradation by the proteosome. It can be rescued by incubation with either chemical chaperones such as phenyl butyrate or glycerol, although the rescued protein has a reduced half-life in the plasma membrane and reduced channel-gating after cAMP stimulation. Heterozygotes with one wild type CFTR allele are asymptomatic and it is believed that only 10-15% of the ER-retained ΔF508 CFTR needs to be rescued to provide therapeutic benefit. Hence, therapies that even partially correct the effects of this mutation will benefit most CF patients.

### SUMMARY

The invention is based on compounds that increase ion transport in mutant-cystic fibrosis transmembrane conductance regulator protein, e.g., ΔF508-CFTR. Such compounds find use in methods of treatment of mutant-CFTR-mediated diseases and conditions, e.g., CF.

The compounds of this invention, which are related to latonduine, have been found to be active using a robust high throughput cell-based assay system. These latonduine analogs increase the level of CFTR at the cell surface. Further the CFTR that is translocated to the cell surface forms functional ion channels. These results obtained in BHK cells were confirmed in functional studies of a human airway epithelial cell-line derived from a CF patient homozygous for ΔF508.

In illustrative embodiments of the present invention, there is provided, use of a compound having a structure of Formula I for treatment of Cystic Fibrosis or for correcting aberrant cellular processing of a mutant cystic fibrosis transmembrane conductance regulator protein, wherein Formula I is:

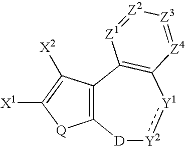

wherein, X¹ and X² are independently selected from the group consisting of: H, F, Cl, Br, I, and R; or alternately, X¹ and X² are linked to form a six-membered aromatic ring; Q is NH, NR, O, or S; D is CO, CH₂, CHR, CR₂, or CS; Y¹ and Y² are independently selected from the group consisting of: N, NH, NR, O, S, CR, CH₂, CHR, and CR₂; Z¹, Z², Z³, and Z⁴ are independently selected from the group consisting of: CR, COH, CO, NH, N, and CNR₂; R is H or a saturated or unsaturated linear, branched or cyclic C₁-C₂₀ alkyl optionally substituted with O, S, N, NH, NR′, OH, F, Cl, Br, I, NH₂, NHR′, NR′₂, SH, or SR′; and R′ is a saturated or unsaturated linear, branched or cyclic C₁-C₁₀ alkyl. The six-membered aromatic ring containing X¹ and X² may comprise nitrogen atoms in addition to the carbon atoms.

In illustrative embodiments of the present invention, there is provided, use of a compound having a structure of Formula I for preparation of a medicament for treating a subject having Cystic Fibrosis or correcting aberrant cellular processing of a mutant cystic fibrosis transmembrane conductance regulator protein, wherein Formula I is:

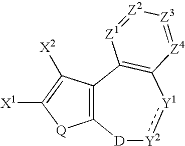

wherein, X¹ and X² are independently selected from the group consisting of: H, F, Cl, Br, I, and R; or alternately, X¹ and X² are linked to form a six-membered aromatic ring; Q is NH, NR, O, or S; D is CO, CH₂, CHR, CR₂, or CS; Y¹ and Y² are independently selected from the group consisting of: N, NH, NR, O, S, CR, CH₂, CHR, and CR₂; Z¹, Z², Z³, and Z⁴ are independently selected from the group consisting of: CR, COH, CO, NH, N, and CNR₂; R is H or a saturated or unsaturated linear, branched or cyclic C₁-C₂₀ alkyl optionally substituted with O, S, N, NH, NR′, OH, F, Cl, Br, I, NH₂, NHR′, NR′₂, SH, or SR′; and R′ is a saturated or unsaturated linear, branched or cyclic C₁-C₁₀ alkyl. The six-membered aromatic ring containing X¹ and X² may comprise nitrogen atoms in addition to the carbon atoms.

In illustrative embodiments of the present invention, there is provided a use described herein, wherein D is CO, CH₂, CHR, or CR₂.

In illustrative embodiments of the present invention, there is provided a use described herein, wherein D is CO, CHR, or CR₂.

In illustrative embodiments of the present invention, there is provided a use described herein, wherein D is CO.

In illustrative embodiments of the present invention, there is provided a use described herein, wherein Q is NH, NR, or O.

In illustrative embodiments of the present invention, there is provided a use described herein, wherein Q is NH, or NR.

In illustrative embodiments of the present invention, there is provided a use described herein, wherein Q is NR.

In illustrative embodiments of the present invention, there is provided a use described herein, wherein Z¹, Z², Z³, and Z⁴ are independently selected from the group consisting of: CR, NH, N, and CNR₂.

In illustrative embodiments of the present invention, there is provided a use described herein, wherein Z¹, Z², Z³, and Z⁴ are independently selected from the group consisting of: NH, N, and CNR₂.

In illustrative embodiments of the present invention, there is provided a use described herein, wherein Z¹, and Z³ are independently selected from the group consisting of: NH, and N.

In illustrative embodiments of the present invention, there is provided a use described herein, wherein Z², and Z⁴ are independently selected from the group consisting of: NH and N.

In illustrative embodiments of the present invention, there is provided a use described herein, wherein Y¹ and Y² are independently selected from the group consisting of consisting of: N, NH, NR, O, CR, CH₂, CHR, and CR₂.

In illustrative embodiments of the present invention, there is provided a use described herein, wherein Y¹ and Y² are independently selected from the group consisting of: N, NH, NR, CR, CH₂, CHR, and CR₂.

In illustrative embodiments of the present invention, there is provided a use described herein, wherein Y² is independently selected from the group consisting of: N, NH, and NR.

In illustrative embodiments of the present invention, there is provided a use described herein, wherein Y¹ is independently selected from the group consisting of consisting of: CR, CH₂, CHR, and CR₂.

In illustrative embodiments of the present invention, there is provided a use described herein, wherein Y¹ is independently selected from the group consisting of: CH₂, and CHR.

In illustrative embodiments of the present invention, there is provided a use described herein, wherein Y² is independently selected from the group consisting of: NH and NR.

In illustrative embodiments of the present invention, there is provided a use described herein, wherein X¹ and X² are independently selected from the group consisting of: H, Cl, Br, I, and R; or alternately, X¹ and X² are linked to form a six-membered aromatic ring. The six-membered aromatic ring containing X¹ and X² may comprise nitrogen atoms in addition to the carbon atoms.

In illustrative embodiments of the present invention, there is provided a use described herein, wherein X¹ and X² are independently selected from the group consisting of: H, F, Cl, Br, I, and R.

In illustrative embodiments of the present invention, there is provided a use described herein, wherein X¹ and X² are independently selected from the group consisting of: H, Cl, Br, I, and R.

In illustrative embodiments of the present invention, there is provided a use described herein, wherein X¹ and X² are independently selected from the group consisting of: H, Cl, Br, and R.

In illustrative embodiments of the present invention, there is provided a use described herein, wherein X¹ and X² are independently selected from the group consisting of: H and Br.

In illustrative embodiments of the present invention, there is provided, a compound having a structure of Formula I, wherein Formula I is:

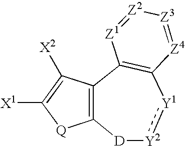

wherein, X¹ and X² are independently selected from the group consisting of: H, F, Cl, Br, I, and R; or alternately, X¹ and X² are linked to form a six-membered aromatic ring; Q is NH, NR, O, or S; D is CO, CH₂, CHR, CR₂, or CS; Y¹ and Y² are independently selected from the group consisting of: N, NH, NR, O, S, CR, CH₂, CHR, and CR₂; Z¹, Z², Z³, and Z⁴ are independently selected from the group consisting of: CR, COH, CO, NH, N, and CNR₂; R is H or a saturated or unsaturated linear, branched or cyclic C₁-C₂₀ alkyl optionally substituted with O, S, N, NH, NR′, OH, F, Cl, Br, I, NH₂, NHR′, NR′₂, SH, or SR′; R′ is a saturated or unsaturated linear, branched or cyclic C₁-C₁₀ alkyl; wherein: when Q is NH, D is CO, Y¹ is CH, Y² is NH, and Z¹ is CR, then either i) R is not H, CO₂H, CO₂Et, or CO₂Me or ii) at least one of Z¹, Z², Z³, and Z⁴ is NH or N; and when Q is NH, D is CO, Y¹ is CH, Y² is NH, and Z² is CR or CNR₂, then either i) R is not NH₂ or H; or at least one of Z¹, Z², Z³, and Z⁴ is NH or N. The six-membered aromatic ring containing X¹ and X² may comprise nitrogen atoms in addition to the carbon atoms.

In illustrative embodiments of the present invention, there is provided a compound described herein, wherein D is CO, CH₂, CHR, or CR₂.

In illustrative embodiments of the present invention, there is provided a compound described herein, wherein D is CO, CHR, or CR₂.

In illustrative embodiments of the present invention, there is provided a compound described herein, wherein D is CO.

In illustrative embodiments of the present invention, there is provided a compound described herein, wherein Q is NH, NR, or O.

In illustrative embodiments of the present invention, there is provided a compound described herein, wherein Q is NH, or NR.

In illustrative embodiments of the present invention, there is provided a compound described herein, wherein Q is NR.

In illustrative embodiments of the present invention, there is provided a compound described herein, wherein Z¹, Z², Z³, and Z⁴ are independently selected from the group consisting of: CR, NH, N, and CNR₂.

In illustrative embodiments of the present invention, there is provided a compound described herein, wherein Z¹, Z², Z³, and Z⁴ are independently selected from the group consisting of: NH, N, and CNR₂.

In illustrative embodiments of the present invention, there is provided a compound described herein, wherein Z¹, and Z³ are independently selected from the group consisting of: NH, and N.

In illustrative embodiments of the present invention, there is provided a compound described herein, wherein Z², and Z⁴ are independently selected from the group consisting of: NH and N.

In illustrative embodiments of the present invention, there is provided a compound described herein, wherein, at least one of Z¹, Z², Z³, and Z⁴ is N.

In illustrative embodiments of the present invention, there is provided a compound described herein, wherein Y¹ and Y² are independently selected from the group consisting of: N, NH, NR, O, CR, CH₂, CHR, and CR₂.

In illustrative embodiments of the present invention, there is provided a compound described herein, wherein Y¹ and Y² are independently selected from the group consisting of: N, NH, NR, CR, CH₂, CHR, and CR₂.

In illustrative embodiments of the present invention, there is provided a compound described herein, wherein Y² is selected from the group consisting of: N, NH, and NR.

In illustrative embodiments of the present invention, there is provided a compound described herein, wherein Y² is selected from the group consisting of: NH and NR.

In illustrative embodiments of the present invention, there is provided a compound described herein, wherein Y¹ is selected from the group consisting of: CR, CH₂, CHR, and CR₂.

In illustrative embodiments of the present invention, there is provided a compound described herein, wherein Y¹ is selected from the group consisting of: CH₂, and CHR.

In illustrative embodiments of the present invention, there is provided a compound described herein, wherein X¹ and X² are independently selected from the group consisting of: H, Cl, Br, I, and R; or alternately, X¹ and X² are linked to form a six-membered aromatic ring. The six-membered aromatic ring containing X¹ and X² may comprise nitrogen atoms in addition to the carbon atoms.

In illustrative embodiments of the present invention, there is provided a compound described herein, wherein X¹ and X² are independently selected from the group consisting of: H, F, Cl, Br, I, and R.

In illustrative embodiments of the present invention, there is provided a compound described herein, wherein X¹ and X² are independently selected from the group consisting of: H, Cl, Br, I, and R.

In illustrative embodiments of the present invention, there is provided a compound described herein, wherein X¹ and X² are independently selected from the group consisting of: H, Cl, Br, and R.

In illustrative embodiments of the present invention, there is provided a compound described herein, wherein X¹ and X² are independently selected from the group consisting of H and Br.

In illustrative embodiments of the present invention, there is provided a compound described herein, for use in treating a subject having Cystic Fibrosis.

In illustrative embodiments of the present invention, there is provided a compound described herein, for use in correcting aberrant cellular processing of a mutant cystic fibrosis transmembrane conductance regulator protein.

In illustrative embodiments of the present invention, there is provided a composition comprising a compound and a pharmaceutically acceptable excipient.

In illustrative embodiments of the present invention, there is provided a method for treating a subject having Cystic Fibrosis or a subject in need of correcting aberrant cellular processing of a mutant cystic fibrosis transmembrane conductance regulator protein, the method comprising administering an effective amount of a compound having a structure of Formula I to a subject in need thereof, wherein Formula I is:

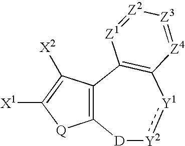

wherein, X¹ and X² are independently selected from the group consisting of: H, F, Cl, Br, I, and R; or alternately, X¹ and X² are linked to form a six-membered aromatic ring; Q is NH, NR, O, or S; D is CO, CH₂, CHR, CR₂, or CS; Y¹ and Y² are independently selected from the group consisting of: N, NH, NR, O, S, CR, CH₂, CHR, and CR₂; Z¹, Z², Z³, and Z⁴ are independently selected from the group consisting of: CR, COH, CO, NH, N, and CNR₂; R is H or a saturated or unsaturated linear, branched or cyclic C₁-C₂₀ alkyl optionally substituted with O, S, N, NH, NR′, OH, F, Cl, Br, I, NH₂, NHR′, NR′₂, SH, or SR′; and R′ is a saturated or unsaturated linear, branched or cyclic C₁-C₁₀ alkyl. The six-membered aromatic ring containing X¹ and X² may comprise nitrogen atoms in addition to the carbon atoms.

In illustrative embodiments of the present invention, there is provided a method described herein, wherein D is CO, CH₂, CHR, or CR₂.

In illustrative embodiments of the present invention, there is provided a method described herein, wherein D is CO, CHR, or CR₂.

In illustrative embodiments of the present invention, there is provided a method described herein, wherein D is CO.

In illustrative embodiments of the present invention, there is provided a method described herein, wherein Q is NH, NR, or O.

In illustrative embodiments of the present invention, there is provided a method described herein, wherein Q is NH, or NR.

In illustrative embodiments of the present invention, there is provided a method described herein, wherein Q is NR.

In illustrative embodiments of the present invention, there is provided a method described herein, wherein Z¹, Z², Z³, and Z⁴ are independently selected from the group consisting of: CR, NH, N, and CNR₂.

In illustrative embodiments of the present invention, there is provided a method described herein, wherein Z¹, Z², Z³, and Z⁴ are independently selected from the group consisting of: NH, N, and CNR₂.

In illustrative embodiments of the present invention, there is provided a method described herein, wherein Z¹, and Z³, are independently selected from the group consisting of: NH and N.

In illustrative embodiments of the present invention, there is provided a method described herein, wherein Z², and Z⁴ are independently selected from the group consisting of: NH and N.

In illustrative embodiments of the present invention, there is provided a method described herein, wherein Y¹ and Y² are independently selected from the group consisting of: N, NH, NR, O, CR, CH₂, CHR, and CR₂.

In illustrative embodiments of the present invention, there is provided a method described herein, wherein Y¹ and Y² are independently selected from the group consisting of: N, NH, NR, CR, CH₂, CHR, and CR₂.

In illustrative embodiments of the present invention, there is provided a method described herein, wherein Y² is selected from the group consisting of: N, NH, and NR.

In illustrative embodiments of the present invention, there is provided a method described herein, wherein Y² is selected from the group consisting of: NH and NR.

In illustrative embodiments of the present invention, there is provided a method described herein,

wherein Y¹ is selected from the group consisting of: CR, CH₂, CHR, and CR₂.

In illustrative embodiments of the present invention, there is provided a method described herein, wherein Y¹ is selected from the group consisting of: CH₂, and CHR.

In illustrative embodiments of the present invention, there is provided a method described herein, wherein X¹ and X² are independently selected from the group consisting of: H, Cl, Br, I, and R; or alternately, X¹ and X² are linked to form a six-membered aromatic ring. The six-membered aromatic ring containing X¹ and X² may comprise nitrogen atoms in addition to the carbon atoms.

In illustrative embodiments of the present invention, there is provided a method described herein, wherein X¹ and X² are independently selected from the group consisting of: H, F, Cl, Br, I, and R.

In illustrative embodiments of the present invention, there is provided a method described herein, wherein X¹ and X² are independently selected from the group consisting of: H, Cl, Br, and R.

In illustrative embodiments of the present invention, there is provided a method described herein, wherein X¹ and X² are independently selected from the group consisting of: H and Br.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

FIG. 1 is a bar graph showing changes in the surface expression of both ΔF508 CFTR (ΔF) and wt CFTR (wt) in BHK cells with 24-hour treatment of 10 μM of the analogues.

FIG. 2 are graphs showing a concentration gradient in the BHK cell screen assay for the latonduines to measure the surface expression of CFTR.

FIG. 3 is a graph showing the amount of surface CFTR quantified by measuring number of pixels of overlap of WGA staining and CFTR.

FIG. 4A illustrates immunoblots used in validation of the latonduines. BHK cells treated for 24 hours with each of the four compounds were harvested and immunoblotted for the presence of the mature band C form of CFTR. FIG. 4B is a graph showing densitometry of the immunoblot to determine the relative amounts of band B and C in the blot.

FIGS. 5A and 5B are graphs showing the effect of the presence of the latonduines on the functionality of CFTR for ΔF508 CFTR (ΔF) in BHK cells cultured at 37° C. as monitored by iodide efflux. Cells were stimulated at time zero with 10 μM Forskolin (Fsk) and 50 μM Genistein (Gst) (SEM; n=3). Further in FIG. 5B, the experiment was also performed on human lung epithelial cells from a cystic fibrosis patient which are expressing ΔF508 CFTR (CFBE).

FIG. 6 is a graph showing changes in the surface expression of both ΔF508 CFTR (F508del) and wt CFTR (wild-type) in BHK cells with 24-hour treatment of 10 μM of the analogues.

### DETAILED DESCRIPTION OF THE INVENTION

Any terms not directly defined herein shall be understood to have the meanings commonly associated with them as understood within the art of the invention. Certain terms are discussed below, or elsewhere in the specification, to provide additional guidance to the practitioner in describing the compositions, devices, methods and the like of embodiments of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples in the specification, including examples of terms, is for illustrative purposes only and does not limit the scope and meaning of the embodiments of the invention herein.

As used herein, the phrase “Cₓ-C_{y} alkyl” is used as it is normally understood to a person of skill in the art and often refers to a chemical entity that has a carbon skeleton or main carbon chain comprising a number from x to y (with all individual integers within the range included, including integers x and y) of carbon atoms. For example a “C₁-C₁₀ alkyl” is a chemical entity that has 1, 2, 3, 4, 5, 6, 7, 8, 9 or 10 carbon atom(s) in its carbon skeleton or main chain.

As used herein, the term “cyclic Cₓ-C_{y} alkyl” is used as it is normally understood to a person of skill in the art and often refers to a compound or a chemical entity in which at least a portion of the carbon skeleton or main chain of the chemical entity is bonded in such a way so as to form a ‘loop’, circle or ring of atoms that are bonded together. The atoms do not have to all be directly bonded to each other, but rather may be directly bonded to as few as two other atoms in the ‘loop’. Non-limiting examples of cyclic alkyls include benzene, toluene, cyclopentane, bisphenol and 1-chloro-3-ethylcyclohexane.

As used herein, the term “linear”, when referring to a chemical entity is used as it is normally understood to a person of skill in the art and often refers to a moiety or a chemical entity in which at the carbon skeleton or main chain of the chemical entity is bonded in such a way so that a ‘loop’ or circle or ring of atoms that are bonded together is not formed within the moiety or chemical entity. Non-limiting examples of liner compounds include methane, ethane, and tert-butane.

As used herein, the term “branched” is used as it is normally understood to a person of skill in the art and often refers to a chemical entity that comprises a skeleton or main chain that splits off into more than one contiguous chain. The portions of the skeleton or main chain that split off in more than one direction may be linear, cyclic or any combination thereof. Non-limiting examples of a branched alkyl are tert-butyl and isopropyl.

As used herein, the term “unbranched” is used as it is normally understood to a person of skill in the art and often refers to a chemical entity that comprises a skeleton or main chain that does not split off into more that one contiguous chain. Non-limiting examples of unbranched alkyls are methyl, ethyl, n-propyl, and n-butyl.

As used herein the term “substituted” is used as it is normally understood to a person of skill in the art and often refers to a chemical entity that has one chemical group replaced with a different chemical group that contains one or more heteroatoms. Often, but not always a substituted alkyl is an alkyl in which one or more carbon atom(s) is/are replaced by one or more atom(s) that is/are not carbon atom(s) and/or one or more hydrogen atom(s) replaced with one or more atom(s) that is/are not hydrogen. For example, chloromethyl is a non-limiting example of a substituted alkyl, more particularly an example of a substituted methyl. Aminoethyl is another non-limiting example of a substituted alkyl, more particularly it is both a substituted propyl and a substituted ethyl.

As used herein the term “unsubstituted” is used as it is normally understood to a person of skill in the art and often refers to a chemical entity that is a hydrocarbon and/or does not contain a heteroatom. Non-limiting examples of unsubstituted alkyls include methyl, ethyl, tert-butyl, and pentyl. In some cases an unsubstituted compound may be a compound that contains a heteroatom, but is not substituted from its original definition. For example an unsubstituted primary amine comprises a primary amine moiety, but otherwise comprises only hydrogen and carbon.

As used herein the term “saturated” when referring to a chemical entity is used as it is normally understood to a person of skill in the art and often refers to a chemical entity that comprises only single bonds. Non-limiting examples of saturated chemical entities include ethane, tert-butyl, and N⁺H₃.

In accordance with one aspect of the invention, there is provided a latonduine analogue compound as described by Formula I, for the treatment of mutant-CFTR-mediated diseases and conditions such as CF:

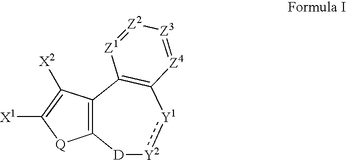

wherein:

X¹ and X² are independently selected from the group: H, Cl, Br, I, and R. Where X is R, X¹ and X² may be linked to form a six-membered aromatic ring. The six-membered aromatic ring containing X¹ and X² may comprise nitrogen atoms in addition to the carbon atoms.

Q is selected from the group comprising: NH, NR, O, and S.

D is selected from the group comprising: CO, CH₂, CHR, CR₂, and CS.

Y¹ and Y² are independently selected from the group comprising: NH, NR, O, S, CR, CH₂, CHR, and CR₂. Y¹ and Y² may be joined by a single or double bond. The presence of either a single or double bond is also denoted in the Formula I above by the presence of the ‘dashed bond’ between Y¹ and Y².

Z¹, Z², Z³, and Z⁴ are independently selected from the group: CR, N, CNR₂, and CNHR.

R is H or a one to ten carbon saturated or unsaturated linear, branched or cyclic alkyl group where carbon atoms may optionally and independently bear substituents including O, OH, F, Cl, Br, I, NH₂, NHR′, NR′₂, SH, and SR′ and/or individual carbon atoms may be replaced with O, S, N, NH, or NR′. R′ is a one to ten carbon saturated or unsaturated linear, branched or cyclic alkyl.

In accordance with another aspect of the invention, there is provided a compound of Formula IIIA or IIIB, for use in the treatment of mutant-CFTR-mediated diseases and conditions such as CF.

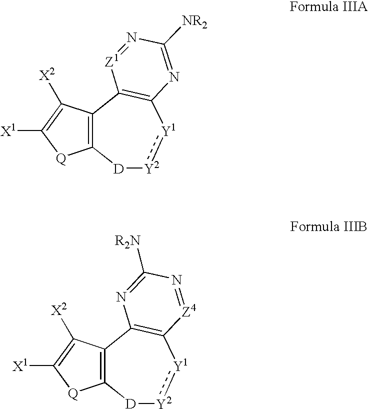

wherein:

X¹ and X² are independently selected from the group: H, Cl, Br, I, and R. Where X is R, X¹ and X² may be linked to form a six-membered aromatic ring. The six-membered aromatic ring containing X¹ and X² may comprise nitrogen atoms in addition to the carbon atoms.

Q is selected from the group comprising: NH, NR, O, and S.

D is selected from the group comprising: CO, CH₂, CHR, CR₂, and CS.

Y¹ and Y² are independently selected from the group comprising: NH, NR, O, S, CR, CH₂, CHR, and CR₂. Further, Y¹ and Y² may be joined by a single or double bond. The presence of either a single or double bond is also denoted in the Formula III above by the presence of the ‘dashed bond’ between Y¹ and Y².

Z¹ and Z⁴ are independently selected from the group: CR, N, CNR₂, and CNHR.

R is H or a one to ten carbon saturated or unsaturated linear, branched or cyclic alkyl group where carbon atoms may optionally and independently bear a substituent including O, OH, F, Cl, Br, I, NH₂, NHR′, NR′₂, SH, and SR′ and individual carbon atoms may be replaced with O, S, N, NH, or NR′. R′ is a one to ten carbon saturated or unsaturated linear, branched or cyclic alkyl.

In accordance with another aspect of the invention, there is provided a compound of Formula IVA or IVB, for use in the treatment of mutant-CFTR-mediated diseases and conditions such as CF.

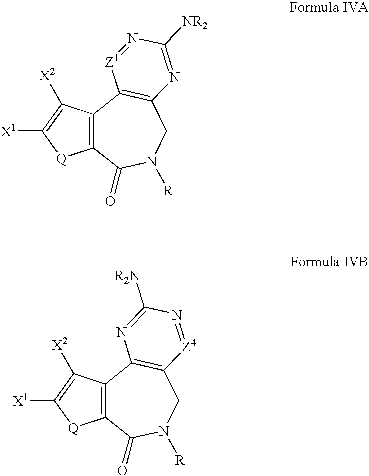

wherein:

X¹ and X² are independently selected from the group: H, Cl, Br, I, and R. Where X is R, X¹ and X² may be linked to form a six-membered aromatic ring. The six-membered aromatic ring containing X¹ and X² may comprise nitrogen atoms in addition to the carbon atoms.

Q is selected from the group comprising: NH, NR, O, and S.

Z¹ and Z⁴ are independently selected from the group: CR, N, CNR₂, and CNHR.

R is H or a one to twenty carbon saturated or unsaturated linear, branched or cyclic alkyl group where carbon atoms may optionally and independently bear substituents including O, OH, F, Cl, Br, I, NH₂, NHR′, NR′₂, SH, and SR′ and individual carbon atoms may be replaced with O, S, N, NH, or NR′. R′ is a one to ten carbon saturated or unsaturated linear, branched or cyclic alkyl.

In accordance with another aspect of the invention, there is provided a latonduine analogue of Formula VA or VB, for use in the treatment of mutant-CFTR-mediated diseases and conditions such as CF.

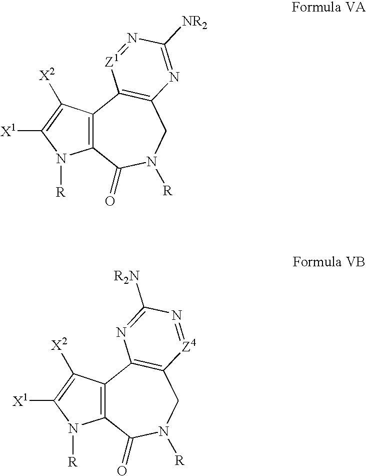

wherein:

X¹ and X² are independently selected from the group: H, Cl, Br, I, and R. Where X is R, X¹ and X² may be linked to form a six-membered aromatic ring. The six-membered aromatic ring containing X¹ and X² may comprise nitrogen atoms in addition to the carbon atoms.

Z¹ and Z⁴ are independently selected from the group: CR, N, CNR₂, and CNHR.

R are independently H or a one to ten carbon saturated or unsaturated linear, branched or cyclic alkyl group where carbon atoms may optionally and independently bear substituents including O, OH, F, Cl, Br, I, NH₂, NHR′, NR′₂, SH, and SW and individual carbon atoms may be replaced with O, S, N, NH, or NR′. R′ is a one to ten carbon saturated or unsaturated linear, branched or cyclic alkyl.

In accordance with another aspect of the invention, there is provided a latonduine analogue of Formula VIA or VIB, for the treatment of mutant-CFTR-mediated diseases and conditions such as CF.

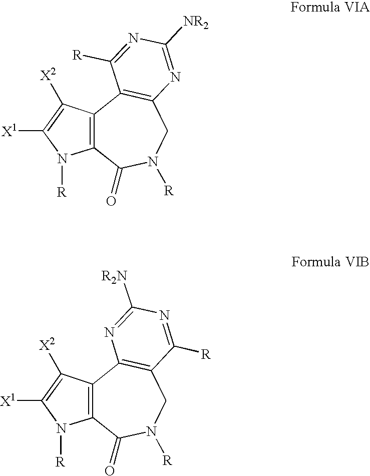

wherein:

X¹ and X² are independently selected from the group: H, Cl, Br, I, and R. Where X is R, X¹ and X² may be linked to form a six-membered aromatic ring. The six-membered aromatic ring containing X¹ and X² may comprise nitrogen atoms in addition to the carbon atoms.

R are independently H or a one to ten carbon saturated or unsaturated linear, branched or cyclic alkyl group where carbon atoms may optionally and independently bear substituents including with O, OH, F, Cl, Br, I, NH₂, NHR′, NR′₂, SH, and SW and individual carbon atoms may be replaced with O, S, N, NH, or NR′. R′ is a one to ten carbon saturated or unsaturated linear, branched or cyclic alkyl.

In accordance with particular aspects of the invention, there are provided the following latonduine compounds for the treatment of mutant-CFTR-mediated diseases and conditions such as CF.

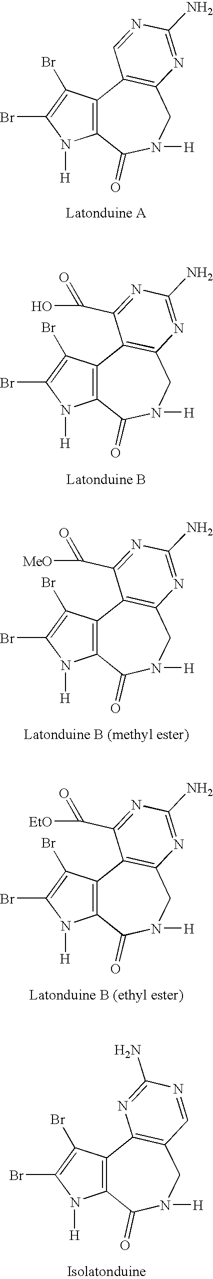

In accordance with another aspect of the invention, there is provided a novel composition of matter according to Formula II.

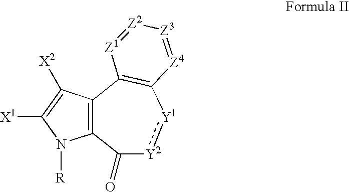

wherein:

X¹ and X² are independently selected from the group: H, Cl, Br, I, and R. Where X is R, X¹ and X² may be linked to form a six-membered aromatic ring. The six-membered aromatic ring containing X¹ and X² may comprise nitrogen atoms in addition to the carbon atoms.

Y¹ is selected from the group comprising: NH, NR, O, S, CR, CH₂, CHR, and CR₂.

Y² is either N or NR. Further, where Y¹ is N or CR and Y² is N, Y¹ and Y² are joined by a double bond.

Z¹, Z², Z³, and Z⁴ are independently selected from the group: CR, N, CNR₂, or CNHR, with the proviso that at least one of Z¹, Z², Z³, and Z⁴ must be N.

R is H or a one to ten carbon saturated or unsaturated linear, branched or cyclic alkyl group where carbon atoms may optionally and independently bear substituents, including O, OH, F, Cl, Br, I, NH₂, NHR′, NR′₂, SH, and SW and individual carbon atoms may be replaced with O, S, N, NH, or NR′. R′ is a one to ten carbon saturated or unsaturated linear, branched or cyclic alkyl.

A novel compound of Formula II is not latonduine A, latonduine B, latonduine B ethyl ester, or latonduine B methyl ester. These compounds were found inactive in cytotoxicity assays against a panel of human cancer lines and for enzyme inhibition against a panel of protein kinases.

The chemical structures of latonduine A and esters thereof have previously been described in Linington et al. (2003) “Latonduines A and B, New Alkaloids Isolated from the Marine Sponge Stylissa carteri: Structure Elucidation, Synthesis, and Biogenetic Implications” Organic Letters, 5: 2735, which is incorporated herein by reference. Analogs of the latonduines have been synthesized and have shown some activity as cytotoxic agents in Putey et al. (2007) “Synthesis of latonduine derivatives via intramolecular Heck reaction” Tetrahedron, 63: 867. Other related compounds are described in: Voskressensky et al. (2006) “A novel synthesis of hexahydroazoninoindoles using activated alkynes in an azepine ring expansion” Tetrahedron, 62: 12392 and Montagne et al. (2005) “Preparation and reactivity of 5-substituted azepino[3,4-b]indoles” J. Heterocyclic Chem. 42: 1433. Methods of preparing or synthesizing compounds of the present invention may be understood by a person of skill in the art having reference to the protocols set out in the Examples, having reference to known chemical synthesis principles, for example those described above, and by using the following scheme. The following scheme provides a generic latonduine analogue synthesis that a person of skill in the art will be able to modify as appropriate to prepare compounds of the present invention.

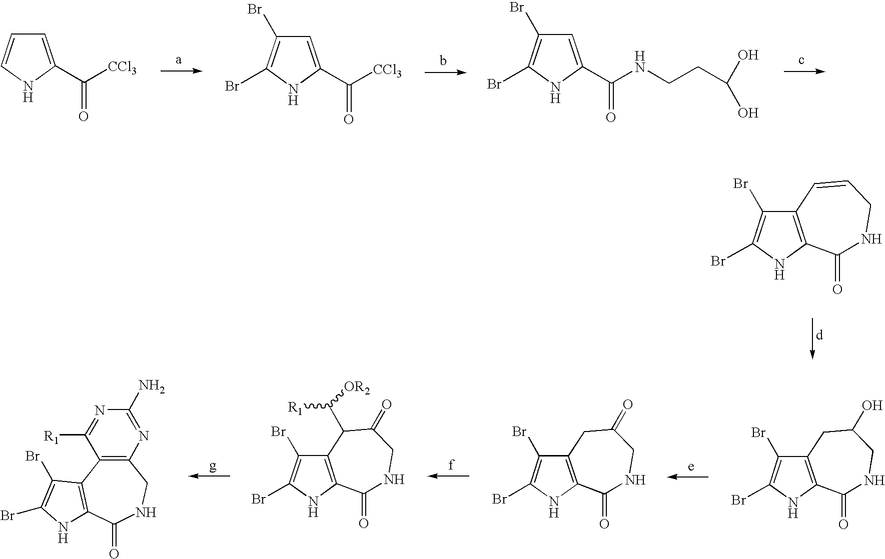

a. Br₂, HOAc; b. 1-amino-3,3-diethoxy propane, MeCN; c. MeSO₃H, Δ; d. i) LiBH₄, BACH-EI, THF, ii) 1 M NaOH, 30% HOOH; e. Dess-Martin periodane, THF; f. R1-orthoester, TFA, Δ; g. guanidine.HCl, K₂CO₃, THF, Δ.

Pharmaceutical compositions according to this invention may be formulated by means known in the art. For example, a compound suitable for parenteral administration may be dissolved in sterile water or saline, or other sterile aqueous media. Compositions suitable for enteric administration may be provided in a liquid, tablet, suspension or gel form. Compounds of this invention may be formulated for timed or sustained release. Compositions suitable for topical administration may be provided as an ointment, cream, gel, liquid, powder, patch or the like. The composition for topical application may further be formulated for timed or sustained release. Various techniques are known to those of skill in the art of formulating pharmaceutical compositions, and may be found in, for example, Remington: the Science & Practice of Pharmacy by Alfonso Gennaro, 20th ed., Williams & Wilkins, (2000).

Compositions in accordance with this invention or for use in this invention may be administered to a patient by standard procedures, including topical, oral, inhalation, intramuscular, intravenous, or intraperitoneal administration. Dosage and duration of treatment may be determined by a practitioner in accordance with standard protocols and information concerning a chosen composition.

In addition to the formulations described previously, the compounds may also be formulated to deliver the active agent by pulmonary means, e.g., administration of an aerosol formulation containing the active agent from, for example, a manual pump spray, nebulizer or pressurized metered-dose inhaler. Suitable formulations of this type can also include other agents, such as antistatic agents, to maintain the disclosed compounds as effective aerosols.

Many compounds of this invention or for use in this invention are generally water soluble and may be formed as salts. In such cases, pharmaceutical compositions in accordance with this invention may comprise a salt of such a compound, preferably a physiologically acceptable salt, which are known in the art. Pharmaceutical preparations will typically comprise one or more carriers acceptable for the mode of administration of the preparation, be it by injection, inhalation, topical administration, lavage, or other modes suitable for the selected treatment. Suitable carriers are those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

An “effective amount” of a pharmaceutical composition according to the invention includes a therapeutically effective amount or a prophylactically effective amount. A “therapeutically effective amount” refers to an amount effective, at dosages and for periods of time necessary, to achieve the desired therapeutic result, such as improved correction of ΔF508 CFTR trafficking or reducing fibrosis. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as improved correction of ΔF508 CFTR trafficking or reducing fibrosis. Typically, a prophylactic dose is used in subjects prior to or at an earlier stage of disease, so that a prophylactically effective amount may be less than a therapeutically effective amount.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD₅₀ (the dose lethal to 50% of the population) and the LD₁₀₀ (the dose lethal to 100% of the population). In some circumstances however, such as in severe disease conditions, it may be necessary to administer substantial excesses of the compositions.

As used herein, a “subject” may be a human, non-human primate, rat, mouse, cow, horse, pig, sheep, goat, dog, cat, etc. The subject may be suspected of having or at risk for having abnormal ΔF508 CFTR trafficking or cystic fibrosis. Diagnostic methods for various abnormal ΔF508 CFTR trafficking disorders and cystic fibrosis and the clinical delineation of abnormal ΔF508 CFTR trafficking disorders and cystic fibrosis diagnoses are known to those of ordinary skill in the art.

Further aspects of the invention relate to treatment of defective chloride ion transport in a subject having CF by use of a compound of this invention.

The following examples are illustrative of some of the embodiments of the invention described herein. These examples should not be considered to limit the spirit or scope of the invention in any way.

### EXAMPLES

### Example I

### Screening Protocol

BHK cells expressing 3HA-tagged ΔF508 CFTR between passages 5-8 were seeded in 96-well plates (Corning half area, black-sided, clear bottom) at 15,000 cells per well and incubated with culture medium for 24 h at 37° C. Each well was then treated with a different test compound (80 compounds per plate) for 24 h at 20 μM final concentration. The remaining 16 wells on each plate were used for control conditions. Cells were fixed in a 2% Para-formaldehyde solution, washed with PBS, and blocked with PBS containing 5% fetal bovine serum (FBS) for 1 h at 4° C.

Blocking solution was replaced with 15 μl of primary antibody solution containing 1% FBS and mouse monoclonal anti-HA antibody (1:150 dilution, Sigma) in PBS. The plates were sealed and left at 4° C. overnight. After three washes with 100 μl PBS, cells were incubated for 1 h with 15 μl of secondary antibody solution containing 1% FBS and anti-mouse IgG conjugated with FITC (1:100 dilution, Sigma) in PBS. Cells were again washed three times with 100 μl of PBS and analyzed in a plate reader (Analyst™ HT96.384, Biosystems) (488 nm excitation, 510 nm emission). Hits were defined as those compounds giving fluorescence at least three standard deviations higher than untreated controls. The mean fluorescence of four untreated wells was used as the background signal when calculating deviations of the 80 compound-treated wells. Hits were then cherry-picked into reservoir plates and re-tested in duplicate using the same assay. Compounds that consistently give signals that were three standard deviations above untreated controls and were not intrinsically fluorescent were considered validated and studied further.

### Example II

### Identification of Latonduines as Correctors of ΔF508-CFTR Misfolding and Trafficking

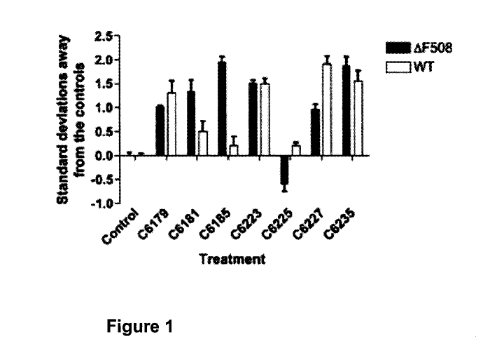

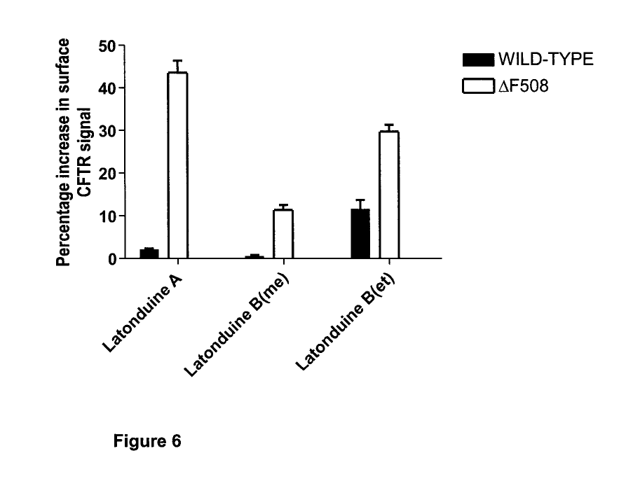

Latonduines A and B were previously isolated from a marine sponge as described in Linington et al. (2003), supra. This citation is hereby incorporated by reference. Chemically synthesizable analogues were assayed using the Screening Protocol and one group, the latonduines, was identified to act as CFTR correctors (see FIGS. 1 and 6). FIG. 1 illustrates that isolatonduine (C6185) gave a positive response. FIG. 6 illustrates changes in the surface expression of both ΔF508 CFTR (F508del) and wt CFTR (wild-type) in BHK cells with 24-hour treatment of 10 μM of the analogues.

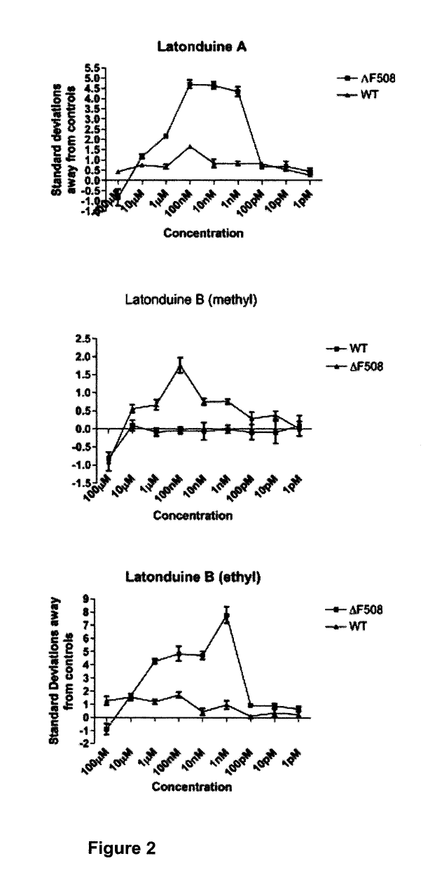

Both latonduine A and the methyl and ethyl esters of latonduine B were tested using the screening protocol at a range of concentrations between 100 μM and 1 pM (FIG. 2). The EC₅₀ for the three latonduines were 0.4 nM, 25 nM and 0.5 nM for latonduines A, B (methyl) and B (ethyl) respectively.

BHK cells expressing F508del-CFTR were treated with 10 μM of the latonduines for 24 hours and then immunostained for CFTR and imaged by confocal microscopy (photomicrograph data not shown).

### Immunofluorescence

Cells were seeded onto 1 cm diameter glass coverslips (5000 per coverslip) and incubated overnight, then treated with compound and fixed in 2% Para formaldehyde. After fixation, cells were blocked using (5% FBS in PBS) for 1 hour at 4° C. The coverslips were then washed in PBS and incubated with primary antibody solution (1% FBS in PBS with 1:200 dilution mouse anti-CFTR antibody (Chemicon) for 2 hours at room temperature (0.05% Tween-20 was added to the blocking solution when staining of intracellular CFTR was required). Coverslips were washed four times in PBS and probed with secondary antibody solution (1% FBS in PBS plus goat anti-mouse Alexa 568 conjugated antibody at 1:1000 dilution) for 1 h at room temperature in the dark. The cells were then washed three times with PBS. The coverslips were then mounted on slides using an antifade mounting solution (Permamount) for confocal microscopy.

Analysis was done using the ImageJ system using 120-150 cells per sample in six fields. The pixels per cell refers to the number of pixels of CFTR stain that overlaps with the cell surface marker stain for each cell in a treatment.

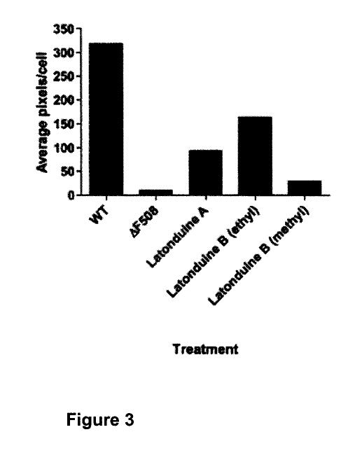

The cell surface was identified using wheat germ agglutinin and the amount of cell surface CFTR measured (FIG. 3). The results demonstrate that the latonduines can all cause trafficking of CFTR to the cell surface. In particular latonduine A and latonduine B (ethyl) produced 29% and 51% respectively of the amount of wild-type CFTR plasma membrane signal.

### Example III

### Immunoblotting Analysis of Latonduines on Biosynthetic Processing of ΔF508-CFTR

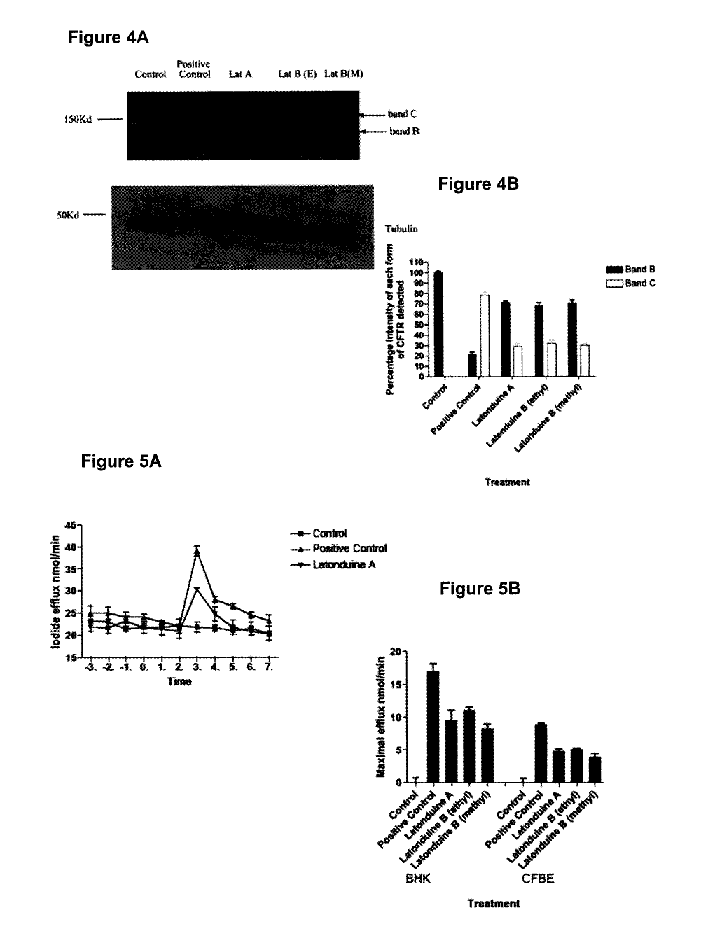

The effects of the latonduines on the biosynthetic processing of ΔF508-CFTR, latonduine treatment was assayed by immunoblotting analysis (FIGS. 4A and B).

Western blots

Cell lysates were quantitated by Bradford assay (BioRad) and separated by SDS-PAGE (6% polyacrylamide gels) and analyzed by Western blotting. Western blots were blocked using 5% skimmed milk in PBS, then were probed overnight at 4° C. with a primary anti-CFTR antibody at a dilution of 1:1000 monoclonal mouse antibody (Chemicon). The blots were washed four times in PBS before the addition of the secondary HRP-conjugated anti-mouse antibody, at a dilution of 1:5000 (Amersham) for one hour at room temperature. The blots were washed five times in PBS and probed for chemiluminescence (Pierce). All samples were run with equal protein loading as determined using the Bradford assay (Biorad). Densitometry of the immunoblots were performed using the ImageJ program.

Bands B and C were detected upon treatment with all three latonduines. In particular, latonduine A partially corrects F508del-CFTR processing and increases surface expression to about 25% of that observed in cells expressing wild-type CFTR. Up to 30% of the F508del-CFTR in cells treated with latonduine A was complex-glycosylated, indicating it had passed through the Golgi. Further it should be noted that latonduine treatment caused an increase in band B but no increase in tubulin expression.

### Example IV

### Iodide Efflux Assay

To determine whether the surface CFTR could form functional ion channels, the BHK cells were treated with latonduines in the iodide efflux assay (FIGS. 5A and 5B).

### Iodide Efflux Assay

Experiments were performed by hand or with a robotic liquid handling system (BioRobot 8000, Qiagen, USA) using Qiagen 4.1 software. Cells were cultured in 24-well plates until they reached confluence in order to perform parallel experiments and comparison analysis. After treatment or not with a test compound, the medium of each well was replaced with 1 ml of iodide loading buffer (in mM: 136 NaI, 3 KNO₃, 2 Ca(NO₃)₂, 11 glucose and 20 Hepes pH 7.4) for 1 hour at 37° C. to permit the I₋ to reach equilibrium. At the beginning of each experiment, the loading buffer was removed by aspiration and cells were washed eight times with efflux buffer (same as loading buffer except that NaI was replaced with 136 mM NaNO₃) to remove extracellular I⁻ in each well. The loss of intracellular I⁻ was determined by removing the medium with efflux buffer every 1 min for up to 11 min. The first four aliquots were recovered at 1-minute intervals in an empty 24-well plate and used to establish a stable baseline in efflux buffer alone. Then, a stimulation buffer (efflux buffer containing 50 μM genistein+10 μM forskolin) was added and samples were also collected every minute in its continued presence. The iodide concentration of each aliquot was determined using an iodide-sensitive electrode (Orion Research Inc., Boston, Mass., USA or Ecomet) and converted to iodide content (i.e. the amount of iodide released during the 1 min interval). Curves were constructed by plotting concentration versus time. Data are presented as means±SEM.

The results demonstrate that all three latonduines act as traffickers of CFTR to generate functional ion channels. The iodide efflux assay was repeated in CFBE cells expressing ΔF508-CFTR. CBFE cells are a human cell line derived from the lung epithelia of a cystic fibrosis patient. The results in FIG. 5B confirm that the latonduines, and in particular latonduine B ethyl ester, can act as correctors of ΔF508-CFTR giving functional ion channels on the cell surface.

### Example V

### Latonduines

The isolation and structure elucidation of latonduine A and the methyl and ethyl esters of latonduine B has been described in Linington et al., 2003, supra. This citation is hereby incorporated by reference.

### Synthesis of Latonduines

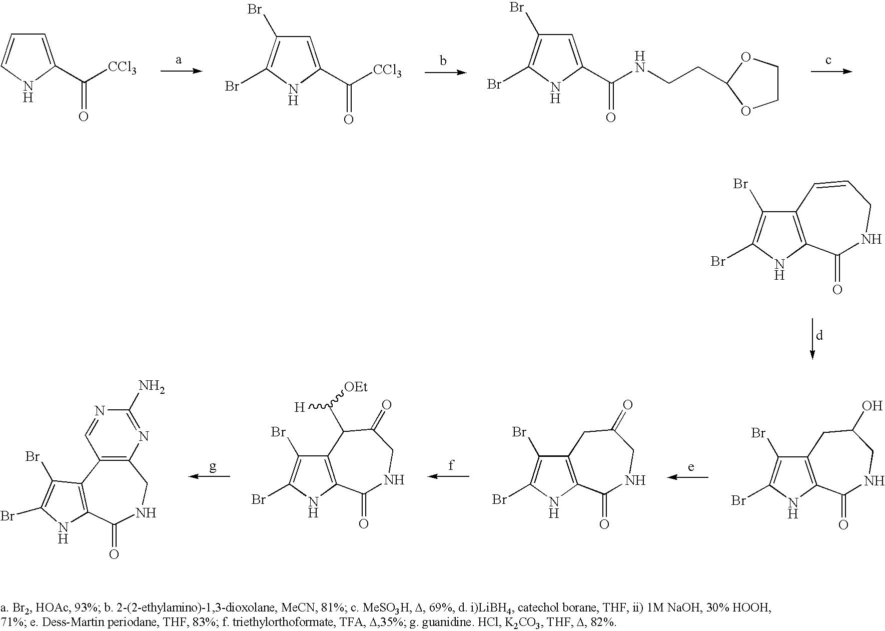

### Isolatonduine Synthesis

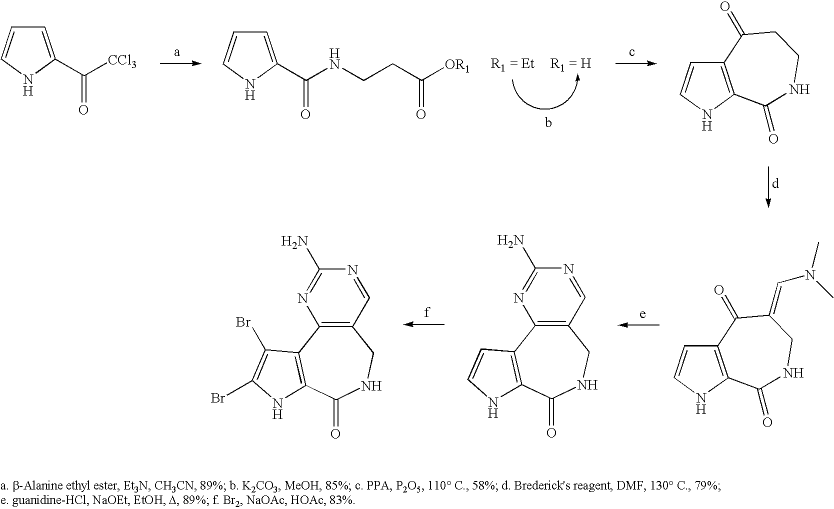

1-28. (canceled)

29. A compound having a structure of Formula I, wherein Formula I is: 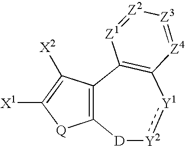
wherein,
X¹ and X² are independently selected from the group consisting of: H, F, Cl, Br, I, and R; or alternately, X¹ and X² are linked to form a six-membered aromatic ring;
Q is NH, NR, O, or S;
D is CO, CH₂, CHR, CR₂, or CS;
Y¹ and Y² are independently selected from the group consisting of: N, NH, NR, O, S, CR, CH₂, CHR, and CR₂;
Z¹, Z², Z³, and Z⁴ are independently selected from the group consisting of: CR, COH, CO, NH, N, and CNR₂;
R is H or a saturated or unsaturated linear, branched or cyclic C₁-C₂₀ alkyl optionally substituted with O, S, N, NH, NR′, OH, F, Cl, Br, I, NH₂, NHR′, NR′₂, SH, or SR′;
R′ is a saturated or unsaturated linear, branched or cyclic C₁-C₁₀ alkyl;
wherein:
when Q is NH, D is CO, Y¹ is CH, Y² is NH, and Z¹ is CR, then either i) R is not H, CO₂H, CO₂Et, or CO₂Me or ii) at least one of Z¹, Z², Z³, and Z⁴ is NH or N; and
when Q is NH, D is CO, Y¹ is CH, Y² is NH, and Z² is CR or CNR₂, then either i) R is not NH₂ or H; or at least one of Z¹, Z², Z³, and Z⁴ is NH or N.

30. The compound of claim 29, wherein:
D is CO, CH₂, CHR, or CR₂;
Q is NH, NR, or O;
Z¹, Z², Z³, and Z⁴ are independently selected from the group consisting of: CR, NH, N, and CNR₂;
Y¹ and Y² are independently selected from the group consisting of: N, NH, NR, O, CR, CH₂, CHR, and CR₂; and
X¹ and X² are independently selected from the group consisting of: H, Cl, Br, I, and R; or alternately, X¹ and X² are linked to form a six-membered aromatic ring.

31. The compound of claim 29, wherein D is CO;
Q is NH;
Z¹, Z², Z³, and Z⁴ are independently selected from the group consisting of: CR, N, and CNR₂;
Y¹ and Y² are independently selected from the group consisting of: NH and CH₂; and
X¹ and X² are independently selected from the group consisting of: H and Br.

32. The compound of claim 29, wherein D is CO.

33. The compound of claim 29, wherein Q is NH.

34-36. (canceled)

37. The compound of claim 29, wherein Z¹, Z², Z³, and Z⁴ are independently selected from the group consisting of: CR N, and CNR₂.

38. The compound of claim 29, wherein Z¹ and Z³ are independently selected from the group consisting of: NH and N.

39. The compound of claim 29, wherein Z² and Z⁴ are independently selected from the group consisting of: NH and N.

40. (canceled)

41. The compound of claim 29, wherein Y¹ and Y² are independently selected from the group consisting of: N, NH, NR, O, CR, CH₂, CHR, and CR₂.

42. (canceled)

43. The compound of claim 29, wherein Y² is selected from the group consisting of: N, NH, and NR.

44. (canceled)

45. The compound of claim 29, wherein Y¹ is selected from the group consisting of: CR, CH₂, CHR, and CR₂.

46. (canceled)

47. The compound of claim 29, wherein X¹ and X² are independently selected from the group consisting of: H, Cl, Br, I, and R; or alternately, X¹ and X² are linked to form a six-membered aromatic ring.

48. The compound of claim 29, wherein X¹ and X² are independently selected from the group consisting of: H, F, Cl, Br, I, and R.

49-50. (canceled)

51. The compound of claim 29, wherein X¹ and X² are independently selected from the group consisting of: H and Br.

52-53. (canceled)

54. A composition comprising a compound according to claim 29 and a pharmaceutically acceptable excipient.

55. A method for treating a subject having Cystic Fibrosis or a subject in need of correcting aberrant cellular processing of a mutant cystic fibrosis transmembrane conductance regulator protein, the method comprising administering an effective amount of a compound having a structure of Formula I to a subject in need thereof, wherein Formula I is: 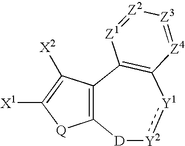
wherein,
X¹ and X² are independently selected from the group consisting of: H, F, Cl, Br, I, and R; or alternately, X¹ and X² are linked to form a six-membered aromatic ring;
Q is NH, NR, O, or S;
D is CO, CH₂, CHR, CR₂, or CS;
Y¹ and Y² are independently selected from the group consisting of: N, NH, NR, O, S, CR, CH₂, CHR, and CR₂;
Z¹, Z², Z³, and Z⁴ are independently selected from the group consisting of: CR, COH, CO, NH, N, and CNR₂;
R is H or a saturated or unsaturated linear, branched or cyclic C₁-C₂₀ alkyl optionally substituted with O, S, N, NH, NR′, OH, F, Cl, Br, I, NH₂, NHR′, NR′₂, SH, or SR′; and
R′ is a saturated or unsaturated linear, branched or cyclic C₁-C₁₀ alkyl.

56. The method of claim 55, wherein
D is CO, CH₂, CHR, or CR₂;
Q is NH, NR, or O;
Z¹, Z², Z³, and Z⁴ are independently selected from the group consisting of: CR, NH, N, and CNR₂;
Y¹ and Y² are independently selected from the group consisting of: N, NH, NR, O, CR, CH₂, CHR, and CR₂; and
X¹ and X² are independently selected from the group consisting of: H, Cl, Br, I, and R; or alternately, X¹ and X² are linked to form a six-membered aromatic ring.

57. The method of claim 55, wherein
D is CO;
Q is NH;
Z¹, Z², Z³, and Z⁴ are independently selected from the group consisting of: CR, N, and CNR₂;
Y¹ and Y² are independently selected from the group consisting of: NH and CH₂; and
X¹ and X² are independently selected from the group consisting of: H and Br.

58-80. (canceled)

81. A method for treating a subject having Cystic Fibrosis or a subject in need of correcting aberrant cellular processing of a mutant cystic fibrosis transmembrane conductance regulator protein, the method comprising administering an effective amount of a compound to a subject in need thereof, wherein the compound is selected from one or more of the following:

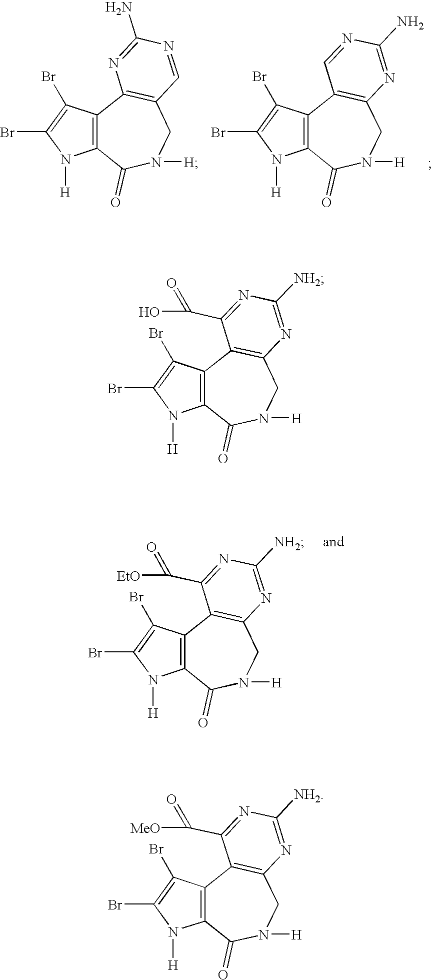

82. The method of claim 81, wherein the compound is selected from one or more of the following: [chemical structure]
